(19) United States
(12) Patent Application Publication
(10) Pub. No.: US 2026/0248477 A1
(43) Pub. Date: Aug. 27, 2026

(54) INTERACTIVE INTERFACE TOOL FOR WAVEFORM VISUALIZATION AND ANNOTATION

(71) Applicant: GE Precision Healthcare LLC, Waukesha, WI (US)

(72) Inventors: Jaydev Kamani, Bengaluru (IN); Rohit Pardasani, Bengaluru (IN); Siddharth Ajith, Bengaluru (IN)

(21) Appl. No.: 19/063,134

(22) Filed: Feb. 25, 2025

(51) Int. Cl. A61B 8/02; A61B 8/00; A61B 8/08

(52) U.S. Cl. CPC A61B 8/02; A61B 8/0866; A61B 8/5223

(57) ABSTRACT

Systems and methods for an interactive interface tool for waveform visualization and annotation are herein provided. In one example, a system is configured to receive manual annotations to a plurality of cardiotocograph datasets via an interactive interface tool; generate, based on the manual annotations to the plurality of cardiotocograph datasets, training data; train a cardiotocograph pattern identification model, using the training data, to identify patterns in cardiotocograph tracings corresponding to defined physiological events associated with hearts of fetuses and uteruses of mothers who correspond to the cardiotocograph tracings during labor; deploy the trained cardiotocograph pattern identification model to identify abnormal patterns in a cardiotocograph waveform sample and generate one or more AI-based annotations for the identified abnormal patterns; and display the one or more AI-based annotations as overlays on the cardiotocograph waveform sample within the interactive interface tool.

### FIELD

Embodiments of the subject matter disclosed herein relate to user interface tools, and more particular to an interactive interface tool for waveform visualization and annotation for use with deep-learning based analytics.

### BACKGROUND

Cardiotocography (CTG) is widely used in pregnancy as a method for assessing fetal well-being, particularly during labor and delivery. CTG records changes in the fetal heart rate (FHR) and their temporal relationship to uterine contractions. The machine used to perform CTG monitoring is called a cardiotocograph, more commonly known as an electronic fetal monitor (EFM). Simultaneous recordings are performed by two separate transducers, one for the measurement of the FHR and a second for measurement of uterine activity (UA) in the context of uterine contractions. The transducers may either be external or internal. The cardiograph recordings are typically in the form of real-time graphical tracings that are either printed on a continuous strip of paper and/or displayed on a graphical display monitor.

The FHR and UA tracings are typically analyzed manually by the obstetric medical team (e.g., including nurses, resident physicians, nurse midwives, attending physicians, etc.) over the course of a labor to identify abnormal patterns and diagnose conditions of the mother and/or fetus. The identification of the patterns helps in assessing various parameters and/or conditions associated with the fetus and/or the mother, such as baseline FHR, contraction duration and frequency, deceleration nature (e.g., late, early, variable), FHR variability, presence of accelerations/decelerations in FHR, and presence of tachysystole. Assessment of these parameters/conditions plays a significant role in identifying fetal abnormalities and the need for intervention during labor.

### BRIEF DESCRIPTION

In one example, a system comprising a processing unit and memory storing instructions executable by the processing unit is configured to receive manual annotations to a plurality of cardiotocograph datasets via an interactive interface tool; generate, based on the manual annotations to the plurality of cardiotocograph datasets, training data; train a cardiotocograph pattern identification model, using the training data, to identify patterns in cardiotocograph tracings corresponding to defined physiological events associated with hearts of fetuses and uteruses of mothers who correspond to the cardiotocograph tracings during labor; deploy the trained cardiotocograph pattern identification model to identify abnormal patterns in a cardiotocograph waveform sample and generate one or more AI-based annotations for the identified abnormal patterns; and display the one or more AI-based annotations as overlays on the cardiotocograph waveform sample within the interactive interface tool.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example system for an interactive interface tool for fetal heart rate (FHR) and uterine activity (UA) analytics;

FIG. 2 shows a training system for a cardiotocograph pattern identification model facilitated by the interactive interface tool;

FIG. 3 shows an example user interface of the interactive interface tool;

FIG. 4 shows an example user interface for user-based annotations of the interactive interface tool;

FIG. 5 shows an example user interface of the interactive interface tool with AI-generated annotations;

FIG. 6 shows an example user interface of the interactive interface tool with AI-generated annotations and user-edits;

FIG. 7 shows a flowchart illustrating method for training the cardiotocograph pattern identification model with training data generated using the interactive interface tool;

FIG. 8 shows a flowchart illustrating a method for generating annotations with the cardiotocograph pattern identification model;

FIG. 9 shows a flowchart illustrating a method for generating the training data using the interactive interface tool; and

FIG. 10 shows a flowchart illustrating a method for updating the cardiotocograph patterns identification model based on user inputted edits to AI-generated annotations.

### DETAILED DESCRIPTION

The following description relates to various embodiments of an interactive interface tool for fetal heart rate (FHA) and uterine activity (UA) analytics. In particular, a system including an interactive interface tool is configured to display FHA and UA waveform data in a user interface, generate training data from user-based annotations to the interactive interface tool, display AI-generated annotations to the waveform data in the user interface, and train and/or update an AI-based model for annotation of the waveform data based on the training data and/or user inputted edits to the AI-generated annotations.

FHR-UA data, in the form of tracings, are typically analyzed manually using printed graphical tracings or tracings presented in graphical user interfaces generated in real-time over the course of labor to identify abnormal patterns. However, manual visual assessment of the graphical tracings requires extensive expertise and may be biased due to the level or type of experience of the provider analyzing the tracing. Further, many medical facilities lack the personnel with sufficient expertise to evaluate tracings and due to the continuous nature of data acquisition and the resulting large volume of information generated by the cardiotocograph device, manual assessment can often lead to practitioners missing identification of abnormal patterns. Although some automated methods for assessing FHR-UA tracings have been developed, these methods are based solely on guidelines and definitions for identifying patterns and conditions expressed in terms of mathematical rules. The existing computational/algorithmic methods are thus statistical and rule-based and not based on the experience of clinicians and thus less appropriate in real life scenarios.

The present disclosure describes an interactive tool for FHR-UA analytics. The interactive interface tool is configured to display FHR and UA waveforms in a temporally aligned fashion, including either real-time acquired data or historical/retrospective data objected for a data repository. The interactive interface tool allows users, such as clinicians, nurses, and other experts, to manually annotate the waveforms to identify particular patterns therein, including regions corresponding to accelerations and decelerations in FHR and contractions in UA, as well as defining baseline FHR and baseline uterine tone. This manual annotation through the interactive interface tool may then be used as training data to train a cardiotocograph pattern identification model. The cardiotocograph pattern identification model may be a deep-learning or other machine-learning based algorithm that is trained, based on this training data, to ingest FHR-UA tracing data and generate AI-based annotations of the particular patterns.

The interactive interface tool is further configured to display the AI-based annotations on corresponding displayed waveforms. For example, the trained cardiotocograph pattern identification model may be deployed in real-time as cardiotocograph data of a patient is acquired. The corresponding FHR and UA waveforms may be displayed by the interactive interface tool along with the annotations outputted by the model. Users may then view these AI-based annotations for diagnostic purposes. Further, the interactive interface tool is configured to allow users to edit the AI-based annotations and the user-inputted edits may be fed back into the model training system to update the trained model as a human-in-the-loop architecture.

This data driven approach thus involves training the machine-learning model using a supervised machine-learning process based on annotations generated via the interactive interface tool. Once trained, the pattern identification model can be deployed in real-life settings to automatically detect occurrence of the defined physiological events based on cardiotocograph data captured for a mother and fetus in real-time. The model performance may be repeatedly refined via the human-in-the-loop architecture whereby experts can edit or otherwise correct AI-outputted annotations. This human-in-the-loop architecture may reduce the need to repeatedly retrain models and may allow for maintaining an up to date and high performance model.

The interactive interface tool herein disclosed provides a single tool for generating training data to train a cardiotocograph pattern identification model, displaying AI-based annotations when generated by the trained cardiotocograph pattern identification model, and generating user-inputted edits for updating the trained cardiotocograph pattern identification model. With this single tool, the computing system may centralize various processes, reducing storage of multiple tools/interface and increasing processing efficiency. Further, as the interface facilitates human-in-the-loop feedback, the user may be able to tailor model outputs to their preferences and specific patient populations.

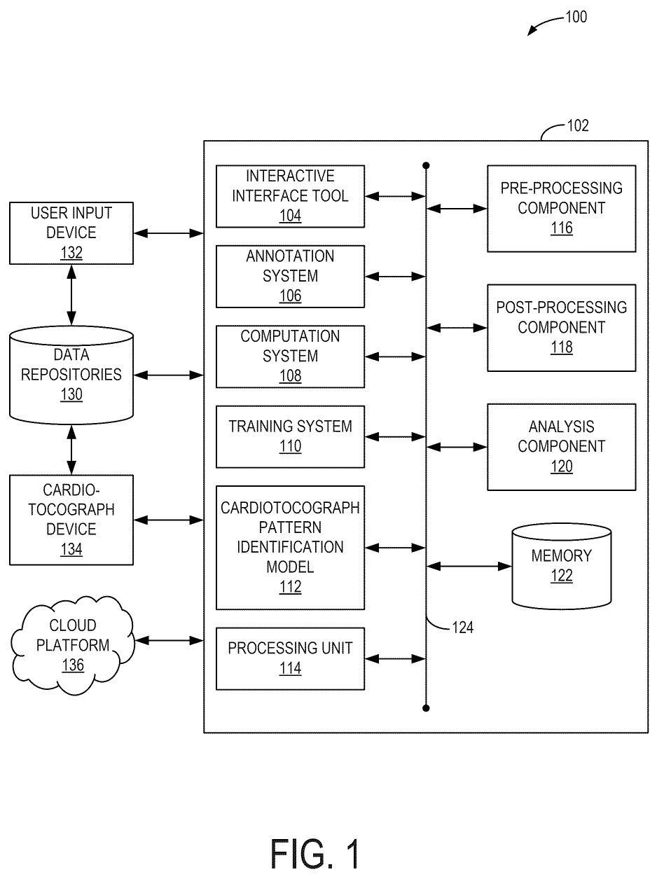

Turning now to FIG. 1, an example system 100 that facilitates FHR and UA analytics using machine learning techniques in accordance with one or more embodiments of the present disclosure is shown. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

In this regard, system 100 comprises a computing device 102 that includes several computer executable components, including an interactive interface tool 104. These computer executable components additionally include an annotation system 106, a computation system 108, a training system 110, a cardiotocograph pattern identification model 112, a pre-processing component 116, a post-processing component 118, and an analysis component 120. These computer/machine executable components, and others described herein, can be stored in memory associated with the one or more machines (e.g., in memory of the computing device 102). The type of computing device 102 may vary. For example, the computing device 102 may correspond to a personal computing device (e.g., a desktop computer, a laptop computer, a smartphone, etc.), a server device, a mobile device, a virtual device, and so on. The memory can further be operatively couple to at least one processor, such that the components can be executed by the at least one processor to perform the operations described herein. For example, in some embodiments, these computer/machine executable components can be stored in memory 122 which can be coupled to processing unit 114 for execution thereof. The computing device 102 may further comprise a device bus 124 that communicatively and operably couples the various components and elements of the computing device 102 to one another.

The computing device 102 may additionally be operatively and/or communicatively coupled to a user input device 132, one or more data repositories 130, a cardiotocograph device 134, and/or a cloud platform 136. The interactive interface tool 104, in some examples, may be stored as a standalone application on a local machine of the computing device 102. In alternative examples, the interactive interface tool 104 may be a web application hosted on-premises or as part of the cloud platform 136, accessible via the computing device 102. Further, it should be understood that while one cardiotocograph device 134 is herein referenced, the computing device 102 may be communicatively coupled to one or more cardiotocograph devices. For example, a labor and delivery ward of a hospital may be equipped with a plurality of cardiotocograph devices for monitoring of multiple mothers/fetuses at the same time. The computing device 102 may be configured to access each of the plurality of cardiotocograph devices to stream data therefrom, for example based on an identifier (e.g., device name or number) of each device.

The interactive interface tool 104 may be configured to display, via the user input device, cardiotocograph data (e.g., FHR and UA waveforms) obtained from the one or more data repositories 130 and/or the cardiotocograph device 134. The cardiotocograph device 134 may additionally be communicatively coupled to the one or more data repositories 130 for temporary or long term storage of data. For example, the one or more data repositories 130 may comprise one or more of an electronic heal record (EMR) system, a radiology information system (RIS), a picture archiving and communication system (PACS), or the like that is configured to store medical data related to patients. The data acquired by the cardiotocograph device 134 may be acquired in real-time (e.g., without intentional delay) and transmitted to the one or more data repositories 130 for long term storage. The computing device 102 may retrieve retrospective cardiotocograph data of a patient from the one or more data repositories 130 or may retrieve real-time cardiotocograph data of a patient from the cardiotocograph device 134, depending on the intended use/application. For example, for generating training data for the cardiotocograph pattern identification model 112, the computing device 102 may obtain retrospective cardiotocograph data from the one or more data repositories 130 to be displayed by the interactive interface tool 104 for annotation. However, for generating AI-based annotations of cardiotocograph data by deploying the trained cardiotocograph pattern identification model 112, real-time cardiotocograph data may be obtained from the cardiotocograph device 134 as it is acquired.

The interactive interface tool 104 may be displayed within a user interface of the user input device 132. For example, the user input device 132 may comprise a care provider device, such as a desktop computer, laptop computer, mobile device, etc. that includes memory, processors, and a display device. The display device may display the user interface that shows the interactive interface tool 104. The user input device 132 may additionally comprise, in some examples, a keyboard, a mouse, a touchpad, or the like that allows users thereof to provide user inputs to the interactive interface tool 104.

The annotation system 106 may be configured as part of the interactive interface tool 104, in some examples, to allow users to generated annotations of the waveform displayed by the interactive interface tool 104. For example, annotations including selection of sections of a waveform that correspond to FHR accelerations, FHR decelerations, uterine contractions, FHR variability, and the like may be inputted to the interactive interface tool 104 via the annotation system 106.

The computation system 108 may be configured to calculate baseline FHR and baseline uterine tone levels based on the cardiotocograph data that is retrieved and/or the waveform annotations. The annotations that are accounted for by the computation system 108 may be AI-based annotations outputted by the trained cardiotocograph pattern identification model 112 or user-inputted annotations. In some examples, the computation system 108 may be configured to calculate a baseline FHR and/or a baseline uterine tone level based on waveform data that discounts certain areas of the waveform. For example, baseline FHR may be calculated based on waveform data not including sections identified as accelerations or decelerations.

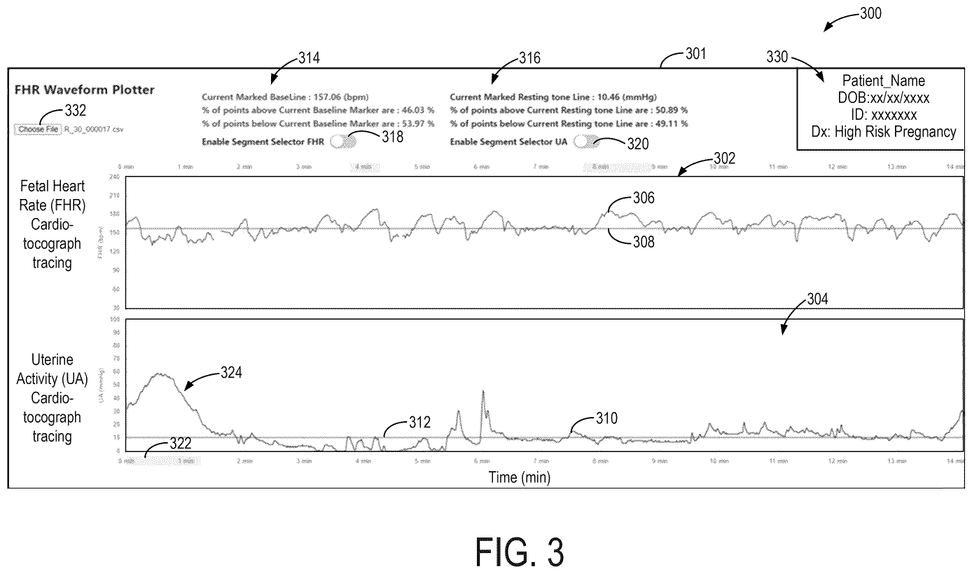

In some examples, the computation system 108 may be additionally configured to calculate percent amounts of waveform data that are greater than and less than the calculated baseline. In some examples, during manual annotation of data for the purpose of generating training data, the computation system 108 may adaptively update calculations of baseline and percent amounts greater than and less than the baseline as the user discounts selected regions (e.g., selects sections of waveform data related to accelerations, decelerations, contractions, and the like). In other examples, during manual annotation of data, the computation system 108 may update calculations of baseline and percent amounts greater than and less than the baseline in response to user inputs to a baseline marker element that is include in the interactive interface tool 104. For example, as will be further described with respect to FIG. 3-6, the baseline marker element may be displayed by the interactive interface tool 104 within the corresponding waveform. The baseline marker element may be a selectable element that the user can manually move to different selected baselines within the waveform. The computation system 108 may identify the selected baseline value and calculate the percent amounts based on the cardiotocograph data and the selected baseline value.

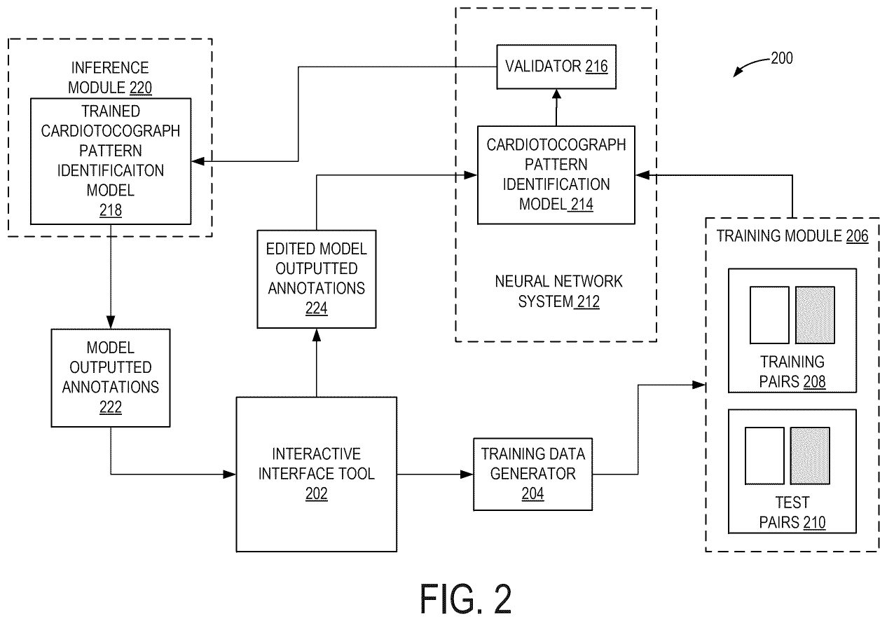

Thus, based on user-inputted annotations to the interactive interface tool 104, the computing device 102 may generate training data of annotations and baseline level computations for the cardiotocograph pattern identification model 112. The training data may include not only the annotations and the patterns the annotations are associated with, but also patient demographic information including age, body mass index, height/weight, comorbidities, fetus size, fetus age, and/or the like that may otherwise affect waveform patterns. The training system 110 may use this training data to train the cardiotocograph pattern identification model 112, as will be further described with respect to FIG. 2. In particular, the training system 110 provides for training and developing the cardiotocograph pattern identification model to identify one or more distinct patterns in cardiotocograph data that correspond to the one or more defined physiological events associated with the fetus and/or mother from which the cardiotocograph data was captured. Although a single model is shown and described herein, it should be appreciated that the number of cardiotocograph pattern identification model 112 can vary.

The type of machine learning model architecture used for the cardiotocograph pattern identification model 112 can also vary. For example, the cardiotocograph pattern identification model 112 can employ various types of machine algorithms, including (but not limited to): deep learning models, neural network models, deep neural network models (DNNs), convolutional neural network models (CNNs), generative adversarial neural network models (GANs), long short-term memory models (LSTMs), attention-based models, transformers, or a combination thereof. In some embodiments, the cardiotocograph pattern identification model 112 can additionally or alternatively employ a statistical-based model, a structural based model, a template matching model, a fuzzy model, or a hybrid, a nearest neighbor model, a naïve Bayes model, a decision tree model, a linear regression model, a k-means clustering model, an association rules model, a q-learning model, a temporal different model, or a combination thereof.

Regardless of the specific type of machine learning model architecture used, the training system 110 may employ a data-driven supervised machine learning process to train and develop the one or more cardiotocograph pattern identification models 112. In a supervised learning framework, the learning system is first given examples of data by which human experts or annotators apply classification labels to a corpus of data (e.g., the user-inputted annotations and associated waveform patterns as inputted to the interactive interface tool 104). The class labels are then used by the learning algorithm to adapt and change its internal, mathematical representations (such as the behavior of artificial neural networks) of the data and mapping to some prediction of classification, etc. The training may comprise iterative methods using numerical optimization techniques that reduce the error between the desired class label and the algorithm's prediction. The newly trained model is then given new data as an input and, if trained well, can classify or otherwise provide assessment of novel data.

In this regard, the training data that is generated via the interactive interface tool includes at least some labeled cardiotocograph data (e.g., labeled via user inputs) annotated with information identifying patterns and their corresponding physiological events. For example, a FHR waveform may be annotated to label a plurality of deceleration patterns. The plurality of deceleration patterns may be noted to indicate a diagnosis such as umbilical cord compression, uterine tachysystole, placental abruption, or other condition associated with frequent decelerations.

Once trained by the training system 110, the cardiotocograph pattern identification model 112 may be deployed to identify patterns within cardiotocograph data and label those patterns. The outputs of the model 112 may be displayed as annotations to the cardiotocograph data when displayed within the interactive interface tool 104. The format of cardiotocograph data supplied as input to the model 112 may be the same as that of the training data or optically transformed into a different machine-readable format by the pre-processing component 116. For example, in some embodiments in which the training data is received by the training system 110 as a digital graphical FHR-UA tracing, the pre-processing component 116 may convert the digital graphical FHR-UA tracing data into its corresponding raw signal data prior to input into the model 112 for training. The pre-processing component 116 can further translate the manually applied ground truth annotation data from the annotated graphical FHR-UA tracings to their corresponding raw signal data segments. Likewise, in some embodiments in which the training data is received as raw signal data, the pre-processing component 116 may convert the raw signal data into a corresponding digital graphical FHR-UA tracing prior to input into the cardiotocograph pattern identification model 112.

As noted above, in some embodiments, the training system 110 can train the cardiotocograph pattern identification model 112 to identify distinct patterns in inputted FHR-UA tracing data samples that represent a window of time, such as 10 minutes or another time interval. In some implementations of these embodiments, the pre-processing component 116 can generate these input data samples prior to processing by the model 112 by cutting/splicing a continuous FHR-UA tracing for a same subject (e.g., same mother/fetus combination) into sequential segments, wherein each segment represents a defined window of time (e.g., each segment corresponding to a 10-minute window of FHR-UA strip data). The pre-processing component 116 can also perform other data pre-processing functions to pre-process the input data prior to input to the model 112 (e.g., during training and/or inferencing). For example, the pre-processing component 116 can pre-process the input data to fill outliers and missing values (e.g., a process known as data cleansing or data engineering). The training system 110 may further employ a supervised machine learning process to train the cardiotocograph pattern identification model 112 to identify the distinct patterns in the input data samples that corresponding to defined physiological events (e.g., FHR acceleration, FHR deceleration, a period of FHR variability, a contract, etc.) using the manually annotated ground truth data associated with at least some of the input data samples.

Additionally, or alternatively, the post-processing component 118 and/or the analysis component 120 can further process and interpret the model output data to generate more accurate and useful results. To facilitate this end, the post-processing component 118 and the analysis component 120 can employ cardiotocograph interpretation domain knowledge and/or cardiotocograph interpretation schema. The cardiotocograph interpretation domain knowledge can include information provided in clinical guidelines, textbooks, articles, and the like that define rules and guidelines for interpreting cardiotocograph data. This information can include aggregated electronic information from various databases and data sources. The cardiotocograph interpretation schema can include similar rules and guidelines for interpreting cardiotocograph data, yet tailored specifically for system 100 (and other systems described herein), for interpreting the results of the cardiotocograph pattern identification model 112. For example, in some embodiments, the cardiotocograph interpretation schema can define rules and/or guidelines for removing spurious results by the post-processing component 118. With these embodiments, the post-processing component 118 can be configured to evaluate the identified patterns and their corresponding physiological events based on defined criteria for valid and invalid patterns provided in the cardiotocograph interpretation schema, wherein the criteria vary for the different physiological events/conditions. For example, the criteria can define thresholds related to timing, duration, frequency, measurement values, and so on. The post-processing component 118 can further remove any identified patterns that fail to pass the defined qualification criteria for the event type from the model results.

The analysis component 120 can also employ the cardiotocograph interpretation domain knowledge and/or the cardiotocograph interpretation schema to further validate whether an identified pattern indicates a corresponding physiological event or condition occurred or not. For example, in some implementations, based on identification of a pattern by the model 112 that correspond to a defined physiological event, the system or analysis component 120 can assume that the event occurred. In other implementations, the analysis component 120 can be configured to perform additional evaluation of the pattern information based on the cardiotocograph interpretation domain knowledge and/or the cardiotocograph interpretation schema to determine with more certainty whether the event or condition did in fact occur. For example, in some implementations, the cardiotocograph interpretation schema can define tailored pattern thresholds for different subjects and clinical contexts based on drugs administered, phase of labor, medical condition of the mother, medical condition of the fetus, medical history of the mother, comorbidities, risk level of the mother, and so on. For instance, fetuses with intrauterine growth restriction are unusually susceptible to the effects of hypoxemia, which tends to progress rapidly, which can cause the thresholds for declaring certain patterns as constituting a hypoxemia related event to be lower in this scenario. Thus, the analysis component 120 can tailor its evaluation of the identified patterns and their correlation to the occurrence of corresponding physiological events or conditions based on other known information about the subject and the clinical context. The analysis component 120 can also aggregate the model output results for sequential input data samples for sequential time segments to generate a timeline of pattern and event information for the subject that spans across a duration of a time. The analysis component 120 can further evaluate the patterns and event information generated by the model longitudinally over time to further clarify the occurrence or non-occurrence of certain events or conditions based on the totality of the aggregated timeline of events and associated patterns.

The analysis component 120 can also employ the cardiotocograph interpretation domain knowledge and/or the cardiotocograph interpretation schema to determine additional information about the physiological state and condition of the mother and/or fetus based on the output of the model 112. For example, in some examples, the analysis component 120 can employ defined rules and schema regarding how to interpret the model identified patterns corresponding to defined events/conditions to determine additional parameters associated with the defined physiological events and/or conditions. For example, in some embodiments, the analysis component 120 can determine the baseline FHR based on the regions of FHR variability determined by the model 112 and using one or more defined baseline FHR algorithmic formulas defined in the cardiotocograph interpretation domain knowledge and/or the cardiotocograph interpretation schema. Additionally, or alternatively, the model 112 may be configured to predict the baseline FHR based on the training data of baseline FHR. The analysis component 120 can also determine various other parameters related to the identified patterns and corresponding physiological events/conditions, including but not limited to: timing of FHR acceleration, duration of FHR acceleration, degree of FHR acceleration, type of FHR acceleration, timing of FHR deceleration, duration of FHR deceleration, degree of FHR deceleration, type of FHR deceleration, timing of FHR period of variability, duration of fetal heart period of variability, degree of FHR variability, contraction timing, contraction duration, contraction frequency, and contraction type. In some implementations of these embodiments, the cardiotocograph interpretation schema can provide the defined rules, algorithms and/or guidelines used by the analysis component 120 to determine (e.g., calculate) or infer this additional information based on the identified patterns and attributes of the patterns, as well as other input variables related to the patient and the clinical context. The analysis component 120 can also aggregate the model output results for sequential input data samples for sequential time segments to generate a timeline of pattern and event information for the subject that spans across a duration of a time. The analysis component 120 can further summarize the condition and/or status of the mother and/or fetus based on the totality of events and conditions up to the current point of time based on information provided in the cardiotocograph interpretation domain knowledge and/or the cardiotocograph interpretation schema.

In some embodiments, the analysis component 120 may employ principles of artificial intelligence to facilitate interpreting the model output data (e.g., the identified patterns and/or their corresponding event classifications) to determine or infer information regarding the clinical condition/state of the mother and/or fetus and/or other relevant parameters associated with the identified patterns and events (e.g., baseline FHR, contraction type, contraction duration, presence of tachysytole, etc.). In certain examples, the analysis component 120 can include a prediction component that employs data (e.g., real-time cardiotocograph data, the model output data, the cardiotocograph interpretation schema, and/or the cardiotocograph interpretation domain knowledge) to monitor a current state of mother and/or fetus. The analysis component 120 can perform interpretation of the cardiotocograph pattern identification model 112 output data explicitly or implicitly. Learning and/or determining interferences by the analysis component 120 can facilitate monitoring of one or more patterns in the patient flow data. For example, the analysis component 120 can employ a probabilistic and/or statistical-based analysis (e.g., factoring into the analysis utilities and costs) to interpret the model output data. The analysis component 120 can employ, for example, an SVM classifier to interpret and classify events and/or conditions associated with the identified patterns. Additionally, or alternatively, the analysis component can employ other classification techniques associated with Bayesian networks, decision trees, and/or probabilistic classification models. Classifiers employed by the analysis component 120 can be explicitly trained (e.g., via a generic training data) as well as implicitly trained (e.g., via observing user behavior, receiving extrinsic information). For example, with respect to SVMs that are well understood, SVMs are configured via a learning or training phase within a classifier constructor and feature selection model. A classifier is a function that maps an input attribute vector, x=(x1, x2, x3, x4, xn), to a confidence that the input belongs to a class—that is, f(x)=confidence(class).

In an aspect, the analysis component 120 can include an inference component that can further enhance automated aspects of the model output interpretation utilizing in part inference-based schemes. The analysis component 120 may employ any suitable machine-learning based techniques, statistical-based techniques and/or probabilistic-based techniques. The analysis component may additionally or alternatively employ a reduced set of factors (e.g., an optimized set of factors) to facilitate providing a most accurate machine learning model for predicting events, conditions and parameters associated with the diagnosis and well-being of the fetus and/or the mother based at least in part on the patterns identified by the cardiotocograph pattern identification model 112. For example, the analysis component 120 may employ expert systems, fuzzy logic, SVMs, HMMs, greedy search algorithms, rule-based systems, Bayesian models (e.g., Bayesian networks), neural networks, other non-linear training techniques, data fusion, utility-based analytical systems, systems employing Bayesian models, etc. In another aspect, the analysis component 120 may perform a set of machine learning computations associated with the one or more patterns in the patient cardiotocograph data identified by the model 112. For example, the analysis component 120 may perform a set of clustering machine learning computations, a set of decision tree machine learning computations, a set of instance-based machine learning computations, a set of regression machine learning computations, a set of regularization machine learning computations, a set of deep Boltzmann machine computations, a set of deep belief network computations, a set of convolutional neural network computations, a set of stacked auto-encoder computations, and/or a set of different machine learning computations. The one or more abnormality parameters, events and/or conditions determined or inferred by the analysis component 120 based at least in part on the output of the model 112 can be reported and stored, for example, in the cardiotocograph interpretation schema and/or the memory 122.

In some examples, the system 100 may be a distributed system, whereby a plurality of users can obtain, display, and interact with cardiotocograph data simultaneously. For example, a first user may stream first cardiotocograph data from a first cardiotocograph device and deploy the trained cardiotocograph pattern identification model to analyze the first cardiotocograph data to generate annotations therefor. The first cardiotocograph data and the generated annotations thereof may be displayed by the interactive interface tool on a first user input device. At the same time, a second user may stream second cardiotocograph data form a second cardiotocograph device and deploy the trained cardiotocograph pattern identification model to analyze the second cardiotocograph data to generate annotations therefor. The second cardiotocograph data and the generated annotations thereof may be displayed by the interactive interface tool on a second user input device at the same time as the first cardiotocograph data is displayed by the first user input device. The system may be adapted to allow for display and interaction with data by multiple users at the same time without decrease in computing performance.

Turning now to FIG. 2, a diagram of a training system 200 for training a cardiotocograph pattern identification model is shown. The training system 200 may be an example of the training system 110 described with respect to FIG. 1 and the cardiotocograph pattern identification model may be the cardiotocograph pattern identification model 112 described with respect to FIG. 1. The cardiotocograph pattern identification model may be a deep learning model, a deep neural network, a convolutional neural network, or the like as described above. The cardiotocograph pattern identification model may be trained to identify the distinct patterns in input data samples that correspond to defined physiological events, wherein the input data samples are cardiotocograph data (e.g., waveforms, tracings, raw data, etc.) for a given period of time, and the distinct patterns correspond to sections of accelerations, decelerations, uterine contractions, variability, and the like in FHR and UA data.

The cardiotocograph pattern identification model 214 may be stored within a neural network system 212. The training system 200 also includes a training module 206, which may be a non-limiting example of the training system 110 of FIG. 1. The training module 206 may include a training dataset comprising a plurality of training pairs of data, such as pairs divided into training pairs 208 and test pairs 210. A number of training pairs 208 and test pairs 210 may be selected to ensure that sufficient training data is available to prevent overfitting, whereby the cardiotocograph pattern identification model 214 learns to map features specific to samples of the training set that are not present in the test set.

Each pair of the training pairs 208 and the test pairs 210 comprises an input and a target. Put simply, the input may be cardiotocograph data, such as tracings, waveforms, raw data, or the like and the target may be pattern labels/annotations of the corresponding data. The training pairs and test pairs may be generated by a training data generator 204. The training data generator 204 may generate sets of training data based on inputs to an interactive interface tool 202. The interactive interface tool 202 may be a non-limiting example of the interactive interface tool 104 described with respect to FIG. 1. The interactive interface tool 202 as herein described may be configured to display cardiotocograph data as FHR and UA waveforms. The interactive interface tool 202 may be configured to allow users to annotate the displayed waveforms, identifying waveform sections associated with various physiological states/waveform patterns, such as FHR accelerations, FHR decelerations, uterine contractions, and more. The training data generator 204 may take these annotations and the corresponding cardiotocograph data and generate the pairs of training data as herein described.

Once each pair is generated, the pair may be assigned to either the training pairs 208 or the test pairs 210. In an embodiment, the pair may be associated to either the training pairs 208 or the test pairs 210 randomly in a pre-established proportion. For example, the pair may be assigned to either the training pairs 208 or the test pairs 210 randomly such that 90% of the pairs generated are assigned to the training pairs, and 10% of the pairs generated are assigned to the test pairs. Alternatively, the pair may be assigned to either the training pairs or the test pairs randomly such that 85% of the pairs generated are assigned to the training pairs, and 15% of the pairs generated are assigned to the test pairs. It should be appreciated that the examples provided herein are for illustrative purposes, and pairs may be assigned to the training pairs 208 dataset or the test pairs 210 dataset via a different procedure and/or in a different proportion without departing from the scope of this disclosure.

The training system 200 may include a validator 216 that validations the performance of the cardiotocograph pattern identification model 214 against the test pairs 210. The validator 216 may take as input a partially trained cardiotocograph pattern identification model 214 and a dataset of test pairs 210, and may output an assessment of the performance of the partially trained cardiotocograph pattern identification model 214 on the dataset of test pairs 210.

Once the cardiotocograph pattern identification model 214 has been validated, a trained cardiotocograph pattern identification model 218 may be used to annotate newly acquired cardiotocograph data by identifying waveform patterns and assigning corresponding labels thereto. The trained cardiotocograph pattern identification model 218 may be stored within an inference module 220. The annotated cardiotocograph data may be outputted as model outputted annotations 222, which are outputted to the interactive interface tool 202 for display. For example, the interactive interface tool 202 may display the cardiotocograph data as FHR and UA waveforms along with the model-outputted annotations.

As described above, the interactive interface tool 202 may be configured for user inputs. For example, a user may input edits, updates, or other changes to the model-generated annotations that are displayed in a user interface. These edited model outputted annotations 224 may be fed back to the cardiotocograph pattern identification model 214 as part of a human-in-the-loop architecture to update the model and model training. In some examples, a user manually saving the file with the user-inputted edits may trigger this feedback loop. From this, the model may learn what changes were made to the outputted annotations to increase performance metrics of the model. The same process of validation may again occur, outputted an updated version of the trained cardiotocograph pattern identification model 218.

The training system 200 herein described may thus be an iteratively updating system that initially trains the cardiotocograph pattern identification model based on training data generated via the interactive interface tool, and then updates the trained cardiotocograph pattern identification model to updated versions based on user-inputted edits to model-outputted annotations inputted to the interactive interface tool. In this way, the interactive interface tool may provide a centralized tool for displaying cardiotocograph data, generating training data, displaying model-generated annotation outputs, and editing the model-generated annotation outputs for improving performance of the model.

Turning now to FIG. 3, an exemplary interactive interface tool 300 is shown. The interactive interface tool 300 may be an example of the interactive interface tool 104 described with respect to FIG. 1. The interactive interface tool 300 may be part of a computing device that includes a processing unit (e.g., processing unit 114) and memory storing instructions executable by the processing unit (e.g., memory 122). The interactive interface tool 300 may be displayed within a user interface 301 on a display device of a user input device that is communicatively coupled to the computing device (e.g., user input device 132). The interactive interface tool 300 may be a standalone application, in some examples, or may be part of a cloud computing platform that is accessible as a web application. The interactive interface tool 300 may comprise a patient demographics panel 330 that includes information of the patient to which the currently displayed cardiotocograph data corresponds, such as a patient name, a date of birth, a medical record number or other identification number, and a relevant diagnosis or admitting diagnosis.

The interactive interface tool 300 comprises an FHR cardiotocograph tracing 302 and a UA cardiotocograph tracing 304 plotted in temporal alignment. For example, the FHR tracing 302 and the UA tracing 304 may share an x-axis that plots time. The FHR tracing 302 may plot FHR in beats per minute (bpm) on its y-axis vs time and the UA tracing 304 may plot uterine activity in millimeters of mercury (mmHg) on its y-axis vs time. The FHR tracing 302 and the UA tracing 304 may be waveform representations of cardiotocograph data obtained from either one or more data repositories or directly from a cardiotocograph device. For example, historical or retrospective data may be obtained from one or more data repositories and real-time data may be obtained from the cardiotocograph device. The FHR tracing 302 and the UA tracing 304 may correspond to a selected waveform sample, which may correspond to a window of time, such as 10 minutes of waveform data. In some examples, the selected waveform sample may be a portion of a larger sample, for example a 10 minute selection of a 30 minute sample, wherein the user can toggle between portions of the 30 minute sample within the interactive interface tool to add annotations as herein described to the 30 minute sample in sections.

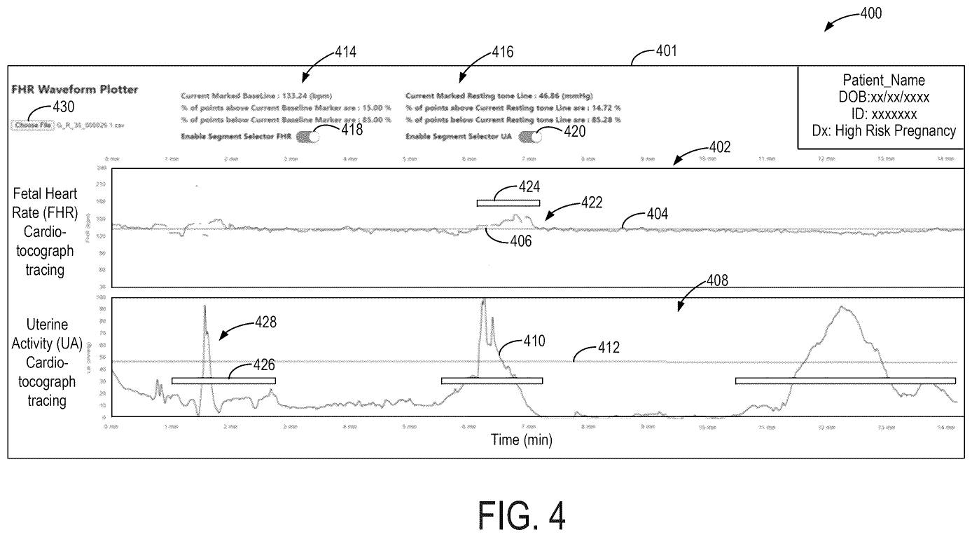

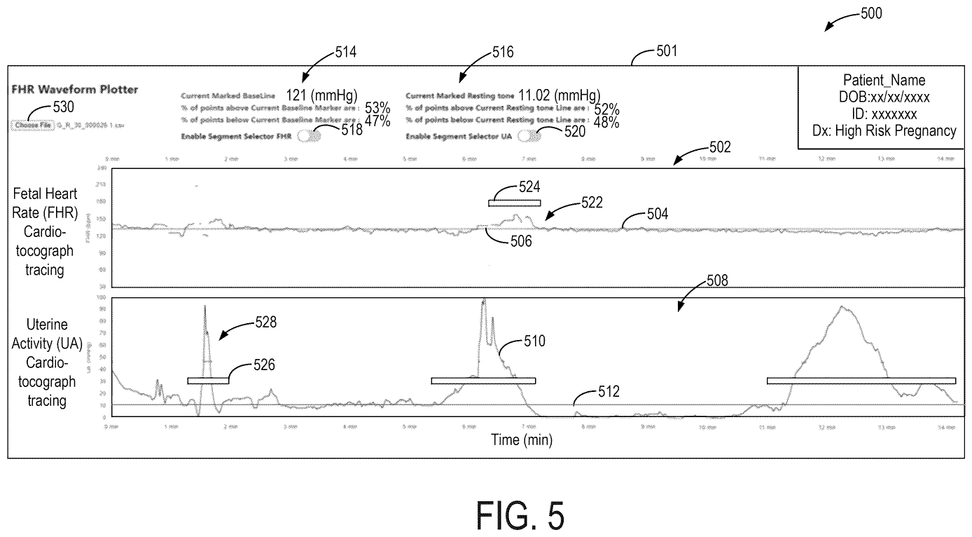

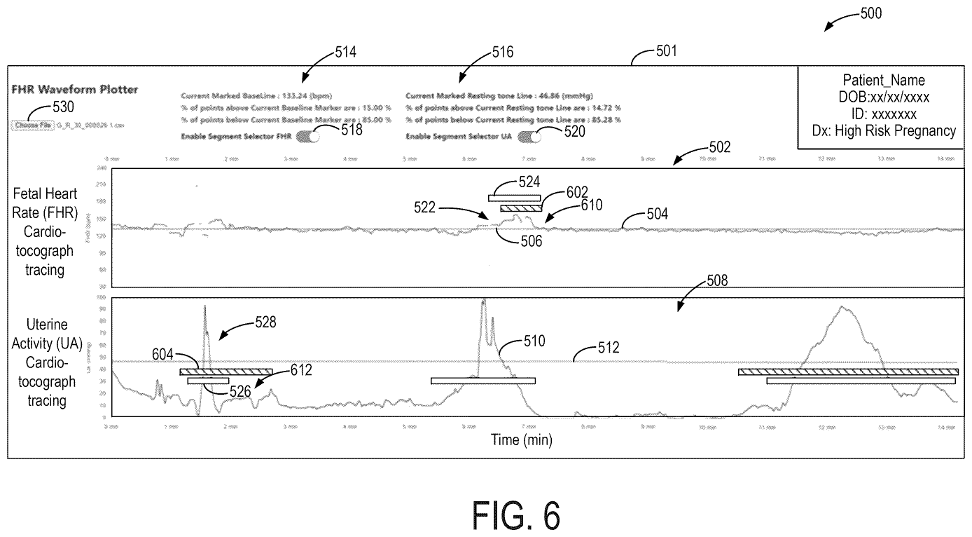

The interactive interface tool 300 may be equipped for various modes. For example, the interactive interface tool 300 may be equipped for manual annotation mode, for example for the purpose of generating data for training a cardiotocograph pattern identification model, as will be further described with respect to FIG. 4. The interactive interface tool 300 may also be equipped for an AI output display mode, wherein the interactive interface tool 300 displays AI-generated annotations, as will be further described with respect to FIG. 5. Further, the interactive interface tool 300 may also be equipped for manual editing of AI-generated annotations, as will be further described with respect to FIG. 6. The interactive interface tool 300 may comprise an FHR selector toggle element 318 and a UA selector toggle element 320. When the FHR selector toggle element 318 is toggled on, the interactive interface tool may be in a manual annotation model for the FHR tracing 302, either for generation of training data when the waveforms are unannotated or for editing of AI-generated annotations. In a similar fashion, when the UA selector toggle element 320 is toggled on, the interactive interface tool 300 may be in a manual annotation model for the UA tracing 304.

The interactive interface tool 300 comprises a file chooser element 332. The file chooser element 332, when selected, may launch a pop-up window that allows the user to select a source of cardiotocograph data. For example, the user may select a data repository such as an EMR, RIS, or PACs as the source. The user may then further select, via the pop-up window, a patient, a timeframe, and the like that identifies the particular waveform they wish to display and annotate. As another example, the user may select a certain cardiotocograph device that corresponds to a desired patient (e.g., is physically coupled to the patient) by serial number, device name or number, or other identification. Cardiotocograph data from that device may then be streamed to the computing device in real-time for display within the interactive interface tool 300.

The FHR tracing 302 may comprise a FHR plot 306 and a baseline FHR marker element 308. The FHR plot 306 may be the waveform of the tracing that shows the FHR along time. The baseline FHR marker element 308 may be a selectable element that marks the baseline FHR value for the given presented waveform sample. Similarly, the UA tracing 304 may comprise a UA plot 310 and a baseline uterine tone marker element 312. The UA plot 310 may be the waveform of the tracing element that shows the UA along time. The baseline uterine tone marker element 312 may be a selectable element that marks the baseline uterine tone value for the given presented waveform sample.

The interactive interface tool 300 may additionally comprise an FHR analytics panel 314 and a UA analytics panel 316. In the example shown in FIG. 3, the FHR analytics panel 314 details a current marked baseline FHR value, determined based on a position of the baseline FHR marker element 308 (and/or based on automated calculation of the baseline), a percentage of waveform points that are above (e.g., greater than) the currently marked baseline value and a percentage of waveform points that are below (e.g., less than) the currently marked baseline value. In other examples, additional analytics, such as a diagnosis, determined patterns in the waveform, other alerts, and the like may also be displayed within the FHR analytics panel 314. Similarly, in the example shown in FIG. 3, the UA analytics panel 316 details a current marked baseline UA (e.g., uterine tone), determined based on a position of the baseline uterine tone marker element 312 (and/or based on automated calculation of the baseline), a percentage of waveform points that are above (e.g., greater than) the currently marked baseline uterine tone value, and a percentage of waveform points that are below (e.g., less than) the currently marked baseline uterine tone value. In other examples, additional analytics, such as a diagnosis, determined patterns in the waveform, other alerts, and the like may also be displayed within the UA analytics panel 316.

Annotation element 322 may be an exemplary annotation to a waveform. Annotation element 322 corresponds to section 324 of the UA tracing 304. As an example, the section 324 may correspond to a uterine contraction during labor. In one example, such as when the interactive interface tool 300 is in a manual mode, the annotation element 322 may be a user-inputted annotation generated in response to user selection of the section 324. In another example, such as when the interactive interface tool 300 is in an AI output display mode, the annotation element 322 may be an AI-based annotation generated by the cardiotocograph pattern identification model.

The annotation element 322 may indicate the type of identified abnormal pattern. For example, the annotation element 322 may be color coded, displayed with a particular fill pattern, or may display text information of the type of abnormal pattern. In other examples, the annotation element 322 may be a selectable element that when selected, such as via a mouse click or mouse hover, may display a pop-up window that details the type of identified abnormal pattern and/or diagnostic indications of the identified abnormal pattern that is determined for the section 324.

It should be understood that the term “abnormal” as herein used refers to a non-baseline pattern and that some such patterns may be considered “normal” or “typical” in a clinical setting based on context, such as a uterine contraction during labor.

In this way, the interactive interface tool 300 as herein described may facilitate display of unannotated cardiotocograph waveform data, annotation of cardiotocograph waveforms for generation of training data for training of the cardiotocograph pattern identification model, display of cardiotocograph waveform data that is annotated by the trained cardiotocograph pattern identification model, and editing of model-generated annotation outputs for updating of the trained cardiotocograph pattern identification model in a single, centralized tool.

Turning now to FIG. 4, an example second interactive interface tool 400 is shown. The second interactive interface tool 400 may be an example of the interactive interface tool 300 when in a manual annotation mode for an unannotated FHR/UA waveform (unannotated in this sense indicating that the waveform is initially presented in the interface without annotations or labels).

Similar to the interactive interface tool 300, the second interactive interface tool 400 is displayed within a user interface 401 and includes an FHR tracing 402 with an FHR plot 404 and a baseline FHR marker element 406 and a UA tracing 408 with a UA plot 410 and a baseline uterine tone marker element 412. The second interactive interface tool 400 additionally comprises an FHR analytics panel 414, a UA analytics panel 416, an FHR segment selector element 418, and a UA segment selector element 420, similar to as described above. The FHR segment selector element 418 and the UA segment selector element 420 may both be toggled on in the example shown, indicating that the interface tool is in manual annotation mode wherein users may select segments of the presented waveforms for annotation.

In some examples, the FHR tracing 402 and the UA tracing 408 may be waveform representations of cardiotocograph data obtained from one or more data repositories, such as EMRs, RISs, PACSs, and/or the like. For example, the cardiotocograph data presented as waveform tracings may be historical or retrospective data that is obtained from the one or more data repositories configured for long term storage of data originally acquired by a cardiotocograph device at a previous time. For example, a file chooser element 430 may be selected to launch a file chooser pop-up panel through which the user may select which FHR/UA waveform sample is to be displayed within the interactive interface tool. In manual mode, as herein described, user-inputted annotations may be taken for generation of training data for a cardiotocograph pattern identification model.

When the displayed waveforms are initially displayed in unannotated form (e.g., prior to receiving user-inputted annotations), the baseline FHR marker element 406 and the baseline uterine tone marker element 412 may initially be displayed by the second interactive interface tool 400 as an average of all points of the corresponding displayed waveform (e.g., as calculated by the computation system 108). However, this average may account for sections that do not correspond to “baseline” values, such as deceleration patterns and acceleration patterns in FHR and contraction patterns in UA, termed herein as abnormal patterns as discussed above. These baseline calculated values may be displayed in the FHR analytics panel 414 and the UA analytics panel 416 along with percent amounts of points that are greater than and less than each calculated baseline value.

The second interactive interface tool 400 may be configured for manual annotation of the displayed waveforms when the selector elements are toggled on. For example, the user may select, by annotation, sections of the waveforms corresponding to abnormal patterns in the waveform data, as will be further described. During manual annotation, the selected sections may be discounted from the calculation of percentage of points above and below the currently marked baseline. For example, in some instances, the marked baseline value may remain unchanged as sections of waveform data are selected and discounted, while the percentages are adaptively recalculated as sections are discounted. Thus, the percentage values of points above and below the currently marked baseline may be adaptively adjusted based on user selections of sections of waveform data during an annotation process. As noted, in some examples, the baseline marker elements may not be adaptive adjusted as sections are discounted, rather the marker elements may be manually adjusted by the user. For example, the baseline FHR marker element 406 and the baseline uterine tone marker element 412 may be selectable elements that the user can select (e.g., via mouse click) to move as they desire to manually chose the current marked baseline. The percentage of points above and below may also be adaptively adjusted in response to manual selection of a baseline value.

In other examples, the position of the baseline marker (e.g., the baseline FHR marker element 406 and the baseline uterine tone marker element 412) may be adaptively adjusted as well. As a non-limiting example, a goal of 50% above the baseline and 50% below the baseline, say for the FHR baseline value, may be pre-set, with a margin of error of ±5%. The position of the corresponding baseline marker element (e.g., the baseline FHR marker element 406) may be automatically adaptively adjusted as the user selects sections of the waveform data, thereby discounting those sections from baseline calculation.

During manual annotation, a user may select sections of the presented waveform samples that correspond to abnormal patterns. For example, a first section 422 of the FHR tracing 402 may correspond to an FHR acceleration. When the user selects the first section 422, a corresponding first annotation 424 may be generated. In some examples, the first annotation 424 may be configurable to indicate the abnormal pattern to which the first section 422 corresponds (E.g., FHR acceleration). For example, the user may select the first annotation 424, such as via a right click, to color or pattern code the first annotation 424, add a text label (e.g., from a list of available abnormal pattern labels), or otherwise add an indication of the type of abnormal pattern. Similarly, a second annotation 426 may be inputted by the user for a second section 428 of the UA tracing 408 corresponding to a uterine contraction. Each annotation that is inputted by the user may include a pattern label as described.

Further, during manual annotation, the baseline and corresponding percentages of points above and below the baseline value may be manually and/or automatically adaptively updated. As an example, when the first annotation 424 is inputted by the user, the points of the first section 422 of the FHR plot 404 to which the first annotation 424 corresponds may be discounted from one or more calculations. For example, the points of the first section 422 may be discounted when the system calculates the percentage of points above and below the currently marked baseline value. Further, in some examples, the currently marked baseline value may be automatically adaptively adjusted in response to discounting the points of the first section 422 from the calculation. In some examples, the baseline marker elements (e.g., the baseline FHR marker element 406 and the baseline uterine tone marker element 412) may be selectable for manual adjustment of the corresponding currently marked baseline value.

In this way, when in manual annotation mode, the interactive interface tool may facilitate user-inputted annotations to sections corresponding to abnormal waveform patterns along with corresponding labels and baseline FHR and uterine tone values. These annotations and labels may be included as part of training data for training a cardiotocograph pattern identification model such that the model is trained in a data-driven manner.

Turning now to FIG. 5, an example third interactive interface tool 500 is shown. The third interactive interface tool 500 may be an example of the interactive interface tool 300 when in an AI output display mode. As is herein descried, when in AI output display mode, AI-generated annotations may be displayed as overlays on corresponding presented FHR/UA tracings.

Similar to the interactive interface tool 300 and the second interactive interface tool 300, the third interactive interface tool 500 is displayed within a user interface 501 and includes an FHR tracing 502 with an FHR plot 504 and a baseline FHR marker element 506 and a UA tracing 508 with a UA plot 510 and a baseline uterine tone marker element 512. The third interactive interface tool 500 additionally comprises an FHR analytics panel 514, a UA analytics panel 516, an FHR segment selector element 518, a UA segment selector element 520, and a file chooser element 530, similar to as described above. The FHR segment selector element 518 and the UA segment selector element 520 may both be toggled off in the example shown, indicating that the interface tool is a display mode, such as AI output display mode wherein AI-generated annotations are displayed.

In some examples, the FHR tracing 502 and the UA tracing 508 may be waveform representations of cardiotocograph data obtained from one or more data repositories, such as EMRs, RISs, PACSs, and/or the like, or directly from a cardiotocograph device. For example, the cardiotocograph data presented as waveform tracings may be real-time data that is obtained from the cardiotocograph device as it is acquired. For example, the file chooser element 530 may be selected to launch a file chooser pop-up panel through which the user may select from which cardiotocograph device data is to be streamed to and displayed within the interactive interface tool.

The third interactive interface tool 500 may include one or more AI-generated annotations, which may be outputted by the cardiotocograph pattern identification model for the cardiotocograph data. The one or more AI-generated annotations may comprise, for example, a first AI-generated annotation 524 and a second AI-generated annotation 526. The first AI-generated annotation 524 may mark a first section 522 of the FHR tracing 502 and the second AI-generated annotation 526 may mark a second section 528 of the UA tracing 508. As a non-limiting example, the first section 522 may be an FHR acceleration pattern and the second section 528 may be a uterine contraction pattern.

In some examples, each of the AI-generated annotations may include an indication of the type of pattern that was identified by the model (e.g., FHR acceleration, FHR deceleration, FHR variability, uterine contraction, and the like). For example, each annotation may be color coded, fill pattern coded, include a text description label, or be a selectable element to launch a pop-up window that includes additional information of the identified pattern.

The FHR analytics panel 514 and the UA analytics panel 516 may each include a current marked baseline value that corresponds to the baseline FHR marker element 506 and the baseline uterine tone marker element 512, respectively. In some examples, the baseline FHR marker element 506 may be positioned for a selected current baseline according to the AI-generated annotations. For example, the system may calculate (e.g., via the computation system 108) the baseline value as an average of all included points of the waveform. The included points of the waveform may not include those points of sections that correspond to abnormal patterns (e.g., the annotated sections), which may be discounted from the calculation. The FHR analytics panel 514 and the UA analytics panel 516 may additionally include percent amounts of points greater than the marked baseline and less than the marked baseline, as previously described.

Turning now to FIG. 6, the third interactive interface tool 500 is again shown. The third interactive interface tool 500 may be in manual annotation mode in the example shown in FIG. 6. For example, the FHR segment selector element 518 and the UA segment selector element 520 may be toggled on in the example shown in FIG. 6. Thus, the third interactive interface 500 may include the AI-generated annotations as described with respect to FIG. 5, but may also be equipped for manual annotation, such as user-inputted edits to the AI-generated annotations for the purpose of providing feedback to the cardiotocograph pattern identification model.

When in manual annotation mode as shown in FIG. 6, the FHR plot 504 and the UA plot 510 may be selectable elements. For example, a user may select sections of the FHR plot 504 that correspond to abnormal patterns in the FHR waveform. A corresponding annotation may be generated in response to user selection of a section, as previously described. When AI-generated annotations are displayed within the interactive interface tool and the interactive interface tool is in manual annotation mode, the manual annotations may be taken as edits to the AI-generated annotations.

As an example, the first AI-generated annotation 524 may be displayed as an overlay on the FHR tracing 502, corresponding to the first section 522 of the FHR plot 504. A first user-inputted annotation 602 may be generated in response to user selection of a third section 610 of the FHR plot 504. The first user-inputted annotation 602 may be a correction to the first AI-generated annotation 524. For example, the first AI-generated annotation 524 may correspond to a first number of points of the FHR plot 504 and the first user-inputted annotation 602 may correspond to a second, smaller number of points of the FHR plot 504, indicating that the first AI-generated annotation 524 included too many points of the FHR plot 504, such as including points that correspond to baseline rather than to an abnormal pattern. In this type of user-inputted edit, the third section 610 may overlap with the first section 522. Other edits are possible, such as indication that the AI-generated annotation corresponds to too little points of the respective plot. Further, user-inputted edits may also include deletions of AI-generated annotations, such as AI-generated annotations that erroneously indicate an abnormal pattern, or additions of annotations to regions of a plot that were not annotated by the cardiotocograph pattern identification model, such as to regions corresponding to abnormal patterns that were missed by the model.

As an example, a second user-inputted annotation 604 may be generated in response to user selection of a fourth section 612 of the UA plot 510. In this example, the fourth section 612 shares points with the second section 528. The fourth section 612 may include more points than the second section 528, indicating that the second AI-generated annotation 526 did not include all relevant points of the corresponding abnormal pattern.

Other types of edits, such as edits to the pattern labels may also be inputted to the interactive interface tool. For example, when annotations are color coded to indicate type of abnormal pattern, the user may change the color of an AI-generated annotation to change the corresponding type of abnormal pattern. Similarly, when annotations are selectable to launch pop-up windows that display additional information of the corresponding waveform section such as the type of abnormal pattern, the user may change the displayed type of abnormal pattern, for example via selectable elements of the pop-up window.

The user-inputted annotations, such as the first user-inputted annotation 602 and the second user-inputted annotation 604, may indicate inaccuracies in the AI-generated outputs. These edits to the AI-generated annotations may be fed back into the training system of the cardiotocograph pattern identification model as part of a human-in-the-loop architecture. In this way, the model may be repeated updated to refine its performance. Further, as the interface facilitates human-in-the-loop feedback, the user may be able to tailor model outputs to their preferences and specific patient populations.

In this way, the interactive interface tool, depending on the particular mode, may allow for generation of training data, display of AI-generated annotations, and editing of AI-generated annotations as part of a human-in-the-loop architecture. Thus, the interactive interface tool provides a single tool for displaying unannotated and/or annotated cardiotocograph waveforms as well as annotating cardiotocograph waveforms for both generating training data and updating an already trained model.

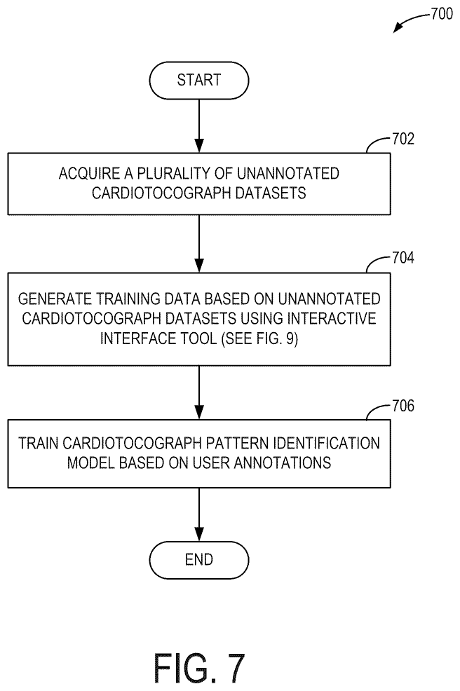

Turning now to FIG. 7, a flowchart illustrating a method 700 for training a cardiotocograph pattern identification model is shown. The cardiotocograph pattern identification model may be a non-limiting example of the cardiotocograph pattern identification model 112 or the cardiotocograph pattern identification model 214 described with respect to FIGS. 1 and 2, according to an exemplary embodiment. Method 700 may be executed by a processor according to instructions stored in memory of a computing system, such as by processing unit 114 according to instructions stored in memory 122 of computing device 102 described with respect to FIG. 1. The cardiotocograph pattern identification model may be trained on training data comprising one or more sets of training pairs comprising an input (e.g., cardiotocograph data) and a target (e.g., annotations of abnormal patterns in the cardiotocograph data).

Method 700 begins at 702, wherein method 700 includes acquiring a plurality of unannotated cardiotocograph datasets. The unannotated cardiotocograph data may comprise cardiotocograph data obtained from a data repository or from a cardiotocograph device. The data repository may be configured to store cardiotocograph data in non-transitory memory, for example the data repository may be an EMR, a PACS, an RIS, or the like. As an example, the unannotated cardiotocograph data acquired for generation of training data may be historical/retrospective cardiotocograph data that was originally acquired by a cardiotocograph device and then transmitted to the data repository for long term storage. In some examples, the acquired unannotated cardiotocograph datasets may comprise a plurality of cardiotocograph waveform samples, each of which may be acquired via and displayed within an interactive interface tool individually.

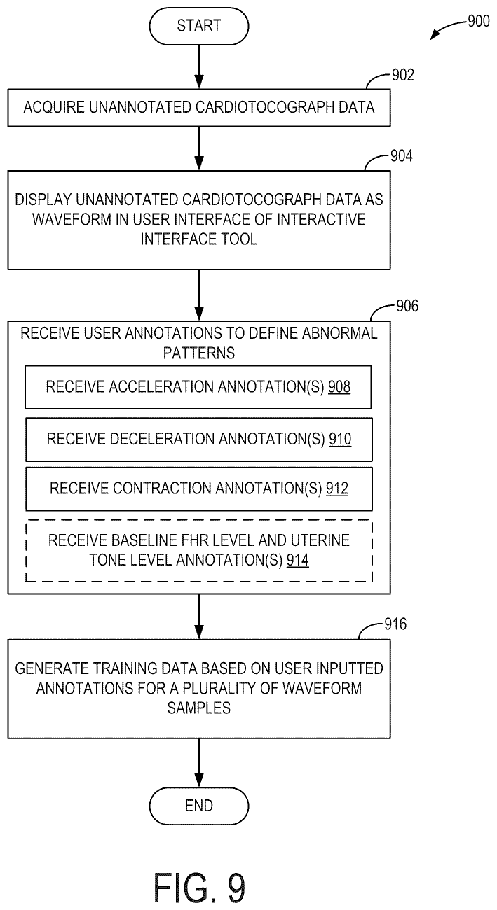

At 704, method 700 includes generating training data based on plurality of unannotated cardiotocograph datasets using the interactive interface tool. As will be further described with respect to FIG. 9, generating training data may comprise receiving user-inputted annotations to the acquired unannotated cardiotocograph datasets through the interactive interface tool. As is described herein, the interactive interface tool, when in manual annotation model, may display FHR and UA tracings of the cardiotocograph waveform data as selectable elements, wherein the user may select sections of the tracings that correspond to abnormal patterns, like FHR decelerations, FHR accelerations, UA contractions, and the like, and label those sections with the corresponding abnormal pattern label. Other portions of training data, such as diagnoses or conditions that correspond to the user-inputted annotations may also be inputted into the interactive interface tool, in some examples.

At 706, method 700 includes training the cardiotocograph pattern identification model based on the user-inputted annotations. In one example, the generated training data may be divided into a training data set and a validation data set. The training data set may be used to train and build the model. Building the model may include applying the input data samples included in the training data set to the model to generate training results. The training results or model output can include information that identifies any distinct patterns in each input data sample (e.g., or no pattern if one is not identified), that correspond to one or more defined physiological events (e.g., FHR acceleration, FHR deceleration, a region of marked variability, and/or a uterine contraction), information classifying the specific physiological event corresponding to each pattern (e.g., the pattern label), and/or information describing/defining attributes or parameters of the identified pattern. For example, the information describing/defining the attributes or parameters of an identified pattern can include information identifying the specific portions of the data in which the one or more patterns are encompassed (e.g., the FHR data, the UA data, or a combination thereof), the start and stop time points of the one or more patterns, and/or the distribution of measurement values that constitute the pattern (e.g., the specific FHR measurements, the specific UA measurements, and/or the combinations thereof). The format of the model output data can vary. For example, the output data can be formatted in a machine-readable format, in a human-readable format, as a graphical FHR-UA tracing with model applied annotation mark-ups (e.g., as shown in FIGS. 5-6), as text, or another format.

Training the model may further include evaluating the loss based on the ground truth annotations (e.g., the user-inputted annotations) applied to the corresponding input data samples (e.g., the acquired unannotated cardiotocograph data). The type of loss function employed can vary. For example, the loss function may be based on mean squared error, cross-entropy, hinge loss, and KL divergence, a distance metric, or the like. The loss function evaluation generation involves determining a measure of difference in accuracy between the training results and the actual ground truth provided by clinical experts via the interactive interface tool. A pattern identified in the training sample may be defined by various metrics that represents the distinct timing and value measurements (e.g., FHR values and/or UA values) that make up the distinct pattern. For example, the pattern can be represented as a graphical distribution of points in space, a reduced dimensionality feature vector, a geometrical shape, a value matrix, or the like. Regardless of the manner in which the pattern is represented/defined, the loss evaluation may involve determining differences between the representation of the pattern in the training results and the ground truth representation for the pattern (or no pattern if ground truth indicates no pattern should have been identified).

In some examples, the loss evaluation may be performed by computing a similarity score between an identified pattern and the corresponding ground truth pattern, such as in a test pair, and determine the degree of loss based on the match similarity score. The similarity score may be computed using one or more statistical techniques and/or one or more artificial intelligence techniques. Alternatively, the similarity score can be computed based on a distance metric, such as a Hamming distance, a Jaccard distance/index, a Dice score/coefficient, or the like.

Based on the loss, one or more model weights and/or parameters may be adjusted. The adjusted model weights and/or parameters may be applied to reduce the amount of loss on the next evaluation of the next training data sample. This process may be performed iteratively until the model loss has stabilized to a defined degree and/or otherwise reached convergence.

Once the cardiotocograph pattern identification model building has progressed to sufficiency on the training data set (e.g., until the model loss has stabilized to a defined degree and/or otherwise reached convergence), the model testing and validation is performed using the validation data set. In this regard, the training component can apply the validation set to the model to generate validation results, which can be evaluated to determine the performance accuracy and specificity of the model. Once the model training has finished, the model is ready to be deployed in clinical context to automatically identify and classify patterns in new cardiotocograph data that correspond to defined physiological events and/or conditions.

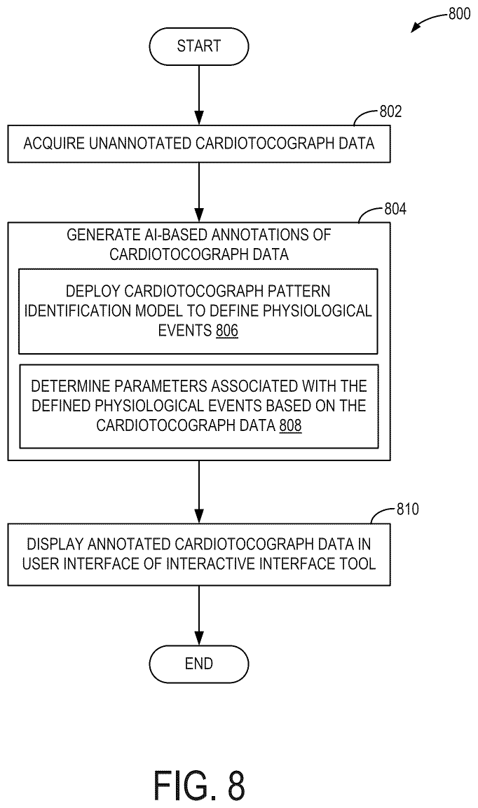

Referring now to FIG. 8, a flowchart illustrating a method 800 for generating AI-based annotations to cardiotocograph data with a trained cardiotocograph pattern identification model. The cardiotocograph pattern identification model may be a non-limiting example of the cardiotocograph pattern identification model 112 or the cardiotocograph pattern identification model 214 described with respect to FIGS. 1 and 2, according to an exemplary embodiment. Method 800 may be executed by a processor according to instructions stored in memory of a computing system, such as by processing unit 114 according to instructions stored in memory 122 of computing device 102 described with respect to FIG. 1. The cardiotocograph pattern identification model may be trained on training data generated via user inputs to an interactive interface tool, as described above.

At 802, method 800 includes acquiring unannotated cardiotocograph data. The unannotated cardiotocograph data may comprise cardiotocograph data obtained from a data repository or from a cardiotocograph device. In one example, the cardiotocograph data may be streamed directly from a cardiotocograph device in real-time, as previously described. In another example, the cardiotocograph data may be acquired from a data repository such as an EMR, RIS, or PACS that stores historical/retrospective cardiotocograph data.

At 804, method 800 includes generating AI-based annotations of the cardiotocograph data. Generating the AI-based annotations for the cardiotocograph data may comprise deploying the trained cardiotocograph pattern identification model to define physiological events, as noted at 806. As described herein, the trained cardiotocograph pattern identification model may be trained to identify abnormal patterns in the unannotated cardiotocograph data. The model output data may include identified patterns or regions in the input data samples (e.g., the acquired cardiotocograph data) that correspond to defined physiological events. For example, in some examples, the cardiotocograph pattern identification model may be configured to directly correlate a pattern in the cardiotocograph data to one or more defined physiological events, such as FHR acceleration, FHR deceleration, a period of FHR variability, or a uterine contraction. In some implementations, in response to detection of an abnormal pattern that corresponds to such an event, an inferencing component of the model can generate an output that indicates that event occurred and the timing of the occurrence. These identified patterns or regions and defined physiological events may be outputted as annotations that are displayed as overlays on the corresponding waveforms of the cardiotocograph data.

In some examples, various post-processing techniques, including removing spurious results, converting the model output data to another format, or the like may be applied to the model output data. The resulting post-processed model output data may be interpreted, for example according to cardiotocograph interpretation schema and/or cardiotocograph interpretation domain knowledge, as previously described with respect to FIG. 1, to correlate the model output data to the defined physiological events and associated parameters (e.g., determining the baseline FHR, determining the contraction duration, determining contraction type, determining tachysystole presence, and so on).

In addition to identification of the physiological events via the cardiotocograph pattern identification model, generating the AI-based annotations may include determining parameters associated with the defined physiological events based on the cardiotocograph data and the patterns, as noted at 808. For example, the parameters may include, but are not limited to: a baseline FHR, timing of FHR acceleration, duration of FHR acceleration, degree of FHR acceleration, type of FHR acceleration, timing of FHR deceleration, duration of FHR deceleration, degree of FHR deceleration, type of FHR deceleration, timing of FHR period of variability, degree of FHR variability, contraction timing, contraction duration, contraction frequency, and contraction type. As an example, the system (e.g., via computation system 108), may calculate a baseline FHR value taking into account the identified abnormal patterns, which as described may be discounted from the calculation of the baseline.

At 810, method 800 includes displaying the annotated cardiotocograph data in a user interface of an interactive interface tool. The FHR and UA waveforms, including tracing plots and generated baseline FHR and/or UA values for the given sample, may be displayed via the interactive interface tool. The generated annotations may be displayed as overlays over the corresponding sections of the FHR and UA plots, as shown in FIGS. 5 and 6.

In some examples, when one or more physiological events are identified via the cardiotocograph pattern identification model and/or the analysis component described above, one or more alerts may be generated based thereon. For example, when a physiological event of placental abruption is identified based on a pattern of frequent FHR decelerations, an alert may be generated indicating to the users that such an event has been identified. In some examples, these alerts may be generated for real-time data (e.g., when the cardiotocograph data is obtained from a cardiotocograph device in real-time).

Turning to FIG. 9, a flowchart illustrating a method 900 for generating model training data using an interactive interface tool. The interactive interface tool may be an example of the interactive interface tool 104 and/or the interactive interface tool 202 described with respect to FIGS. 1 and 2, respectively, according to an exemplary embodiment. Method 900 may be executed by a processor according to instructions stored in memory of a computing system, such as by processing unit 114 according to instructions stored in memory 122 of computing device 102 described with respect to FIG. 1. The cardiotocograph pattern identification model may be trained on training data generated via user inputs to an interactive interface tool, as described above.

At 902, method 900 includes acquiring unannotated cardiotocograph data. The unannotated cardiotocograph data may comprise cardiotocograph data obtained from a data repository or from a cardiotocograph device. The data repository may be configured to store cardiotocograph data in non-transitory memory, for example the data repository may be an EMR, a PACS, an RIS, or the like. As an example, the unannotated cardiotocograph data acquired for generation of training data may be historical/retrospective cardiotocograph data that was originally acquired by a cardiotocograph device and then transmitted to the data repository for long term storage.

At 904, method 900 includes displaying the unannotated cardiotocograph data as a waveform in a user interface of the interactive interface tool. For example, an FHR tracing (e.g., FHR tracing 402 of FIG. 4) and a UA tracing (e.g., UA tracing 408 of FIG. 4) may be displayed. Each tracing may comprise a plot of points. The FHR tracing and the UA tracing may be plotted in a time-aligned manner (e.g., with a shared time axis). In some examples, the acquired unannotated cardiotocograph data may comprise a plurality of cardiotocograph waveform samples, in which case each sample may be displayed, separately, via the interactive interface tool and, as will be described below, annotated to define abnormal patterns of the corresponding displayed waveform sample. A waveform sample, in this context, may be an FHR or UA waveform for a given timeframe (e.g., for 10 minutes, 15 minutes, 30 minutes, etc.) of the acquired cardiotocograph data. In some examples, multiple waveform samples may be included within the same cardiotocograph data. In such an example, each sample may be displayed on its own. The user can toggle between full view or partial view (e.g., a particular 10 minute time window in 30 minutes of waveform). The user can also select one or more of the multiple waveforms to be viewed on the interface.

At 906, method 900 includes receiving user annotations to define abnormal patterns. The user annotations may include, but are not limited to, FHR accelerations, as noted at 908, FHR deceleration annotations, as noted at 910, uterine contraction annotations, as noted at 912, and, optionally, baseline FHR level and baseline uterine tone level annotations, as noted at 914. The user annotations, may include, as a non-limiting example, user selection of one or more sections of a given tracing, such as the FHR tracing or the UA tracing. n some examples, each selected section of a tracing may also be given a pattern identification label thereby defining the physiological event to which the section corresponds, such as FHR deceleration, FHR acceleration, uterine contraction, etc. The user annotations may be received when the interactive interface tool is in a manual annotation mode, such as is described with respect to FIG. 4.

As noted herein, a baseline FHR value and a baseline UA value (e.g., a baseline uterine tone value) may be calculated by the system for the displayed waveform sample initially (e.g., for the unannotated version of the data). This calculation may include calculating the percentage of points of the corresponding plot that are greater than the corresponding defined baseline value and the percentage of points that are less than the corresponding defined baseline value. Then, each selected section of the corresponding tracing may be discounted from the calculation of the percentages of points above and below the baseline. In some examples, as described above, the user may manually move a baseline marker element to manually select a baseline value for the corresponding tracing. In such examples, the percentages of points may be adaptively updated based on the selected baseline value. In other examples, the baseline value for a tracing, and thus the corresponding baseline marker element, may be adaptively updated as the user selects sections of the plots corresponding to abnormal patterns, where a target percentage split above and below the baseline is predefined (e.g., a target of 50% above and 50% below with a predefined margin of error).

At 916, method 900 includes generating training data based on the user inputted annotations to a plurality of waveform samples. As described above, the obtained unannotated cardiotocograph data may comprise a plurality of waveforms, which in some examples may each include a plurality of waveform samples therein. User-inputted annotations defining abnormal patterns and corresponding physiological events may be received for each displayed waveform sample. These defined abnormal patterns and corresponding physiological events as inputted by the user to the interactive interface tool may form, along with the unannotated cardiotocograph data (e.g., waveforms and waveform samples) the training data, which includes unannotated cardiotocograph data as inputs and annotated cardiotocograph data, for example with annotated sections corresponding to abnormal patterns and corresponding physiological event labels, as targets. This training data may then be used to train a cardiotocograph pattern identification model, as described above with respect to FIG. 7.

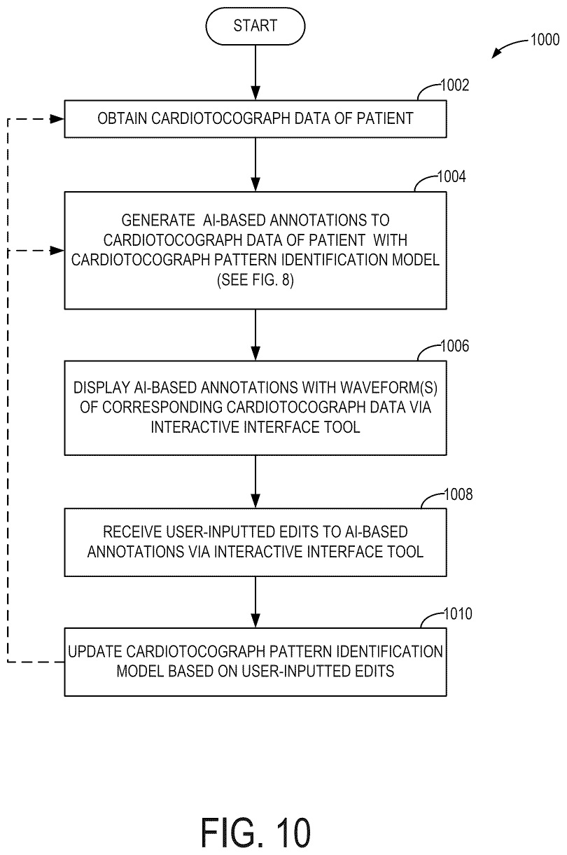

Turning now to FIG. 10, a flowchart illustrating a method for iteratively updating a trained cardiotocograph pattern identification model based on user-inputted edits to model output data such as AI-generated annotations. The user-inputted edits may be received via an interactive interface tool. The interactive interface tool may be an example of the interactive interface tool 104 and/or the interactive interface tool 202 and the cardiotocograph pattern identification model may be an example of the cardiotocograph pattern identification model 112 and/or the trained cardiotocograph pattern identification model 218 described with respect to FIGS. 1 and 2, respectively, according to an exemplary embodiment. Method 900 may be executed by a processor according to instructions stored in memory of a computing system, such as by processing unit 114 according to instructions stored in memory 122 of computing device 102 described with respect to FIG. 1. The cardiotocograph pattern identification model may be trained on training data generated via user inputs to an interactive interface tool, as described above.

At 1002, method 1000 includes obtaining cardiotocograph data of a patient. In some examples, the cardiotocograph data may be a waveform sample (e.g., a first waveform sample) that includes FHR and UA points for a specified amount of time (e.g., 10 minutes). The cardiotocograph data of the patient may be unannotated data, such as raw data. The unannotated cardiotocograph data may comprise cardiotocograph data obtained from a data repository or from a cardiotocograph device. In one example, the cardiotocograph data may be streamed directly from a cardiotocograph device in real-time, as previously described. In another example, the cardiotocograph data may be acquired from a data repository such as an EMR, RIS, or PACS that stores historical/retrospective cardiotocograph data.

At 1004, method 1000 includes generating AI-based annotations for the cardiotocograph data of the patient with a trained cardiotocograph pattern identification model. As described with respect to FIG. 8, generating AI-based annotations with the trained model may comprise identifying waveform patterns that correspond to physiological events, such as FHR accelerations, FHR decelerations, periods of FHR variability, uterine contractions, and the like. Each identified abnormal pattern may correspond to a particular section of one of an FHR tracing and a UA tracing of the cardiotocograph data. Corresponding annotations to those sections may be generated for display within an interactive interface tool, as previously described.

At 1006, method 1000 includes displaying the AI-based annotations with waveform(s) of the corresponding cardiotocograph data via the interactive interface tool. As described with respect to FIGS. 5 and 8, the cardiotocograph data may include an FHR waveform and a UA waveform. Tracing plots thereof may be displayed in a time aligned manner within the interactive interface tool with the corresponding AI-generated annotations, including section annotations and corresponding pattern type identifiers (e.g., color codes, pattern file codes, displayable text description, etc.).

At 1008, method 1000 includes receiving user-inputted edits to the AI-based annotations via the interactive interface tool. As described with respect to FIG. 6, the interactive interface tool may be toggled into a manual annotation mode while the AI-generated annotations are displayed as overlays on the corresponding waveforms. In manual annotation mode, the user may input edits to the displayed AI-generated annotations. For example, the user may change the points of the section to which an AI-generated annotation corresponds, such as by selecting the AI-generated annotation and manually updating the annotation (e.g., by sliding the annotation to include more points or include less points) or by inputting a new annotation that corresponds to points of the plot that overlap with the points of the section of the AI-generated annotation. Additionally, or alternatively, the user may also change the physiological event label of respective annotations, for example to correct an identified event label.

At 1010, method 1000 includes updating the cardiotocograph pattern identification model based on the user-inputted edits. As an example, the user-inputted edits may be fed into the model training system (e.g., the training system 200 of FIG. 2) as part of a human-in-the-loop architecture. The training system may be adapted to update the model based on the user-inputted edits in order to refine the performance of the model.

Once the model is updated, in one example, the method 1000 optionally returns to 1004, in which the updated cardiotocograph pattern identification model repeats generation of AI-based annotations for the cardiotocograph data, specifically to the first waveform sample, where the cardiotocograph data is the same cardiotocograph data to which the user-inputted edits correspond. In another example, the method 1000 optionally returns to 1002 to obtain new cardiotocograph data, such as a second waveform sample. In one example, the new cardiotocograph data may be different from the cardiotocograph data to which the user-inputted edits corresponded. The updated cardiotocograph pattern identification model may then be deployed to analyze the second waveform sample to identify waveform patterns therein that correspond to defined physiological events, as is herein described.

The method 1000 then repeats from 1006 on in a cyclical, iterative manner, wherein, in response to user-inputted edits being received for a model output, those edits are fed back into the training system of the model to iteratively refine the performance of the model based on user-inputted feedback. In this way, the model can be updated in response to user edits rather than needing to fully retrain the model or develop an entirely new model. This may reduce the processing demands for the system overall. Further, the interactive interface tool may thus allow for display of model outputs (e.g., AI-generated annotations) as well as input of user edits to those model outputs for enhancing of the model performance.

A technical effect of the systems and methods provided herein is that the interactive interface tool provides a single, centralized tool for display of cardiotocograph waveform data and manual annotation of cardiotocograph data that enables efficient data processing and subsequent model training. In particular, unannotated cardiotocograph waveform data may be displayed and the tool may facilitate manual annotation of the data, for example for generation of training data. The training data may then the be used to train a cardiotocograph pattern identification model, which may be deployed to identify abnormal patterns in cardiotocograph data. Corresponding annotations generated by the model as at least part of a model output may be displayed within the interactive interface tool. Further, manual annotations may be inputted to the tool in order to edit the AI-generated annotations as part of a human-in-the-loop architecture for improving the performance of the model. Additionally, the model may be a data-driven manner of analyzing cardiotocograph data in real-time, thereby drastically reducing the time and effort spent in manual cardiotocograph data (e.g., strip data or digitally graphically displayed data) assessment time, while also minimizing errors that would result from a rules-based statistical approach.

The interactive interface tool provides specific technical improvements to computer-based cardiotocograph analysis systems. In particular, the system reduces processing overhead and memory usage compared to conventional systems by implementing a single centralized tool that handles multiple functions rather than requiring separate tools and interfaces for training data generation, model deployment, and model updating. The human-in-the-loop architecture allows for efficient incremental model updates without requiring full model retraining, which significantly reduces computational resource requirements. Further, as the interface facilitates human-in-the-loop feedback, the user may be able to tailor model outputs to their preferences and specific patient populations.

The system implements specialized data pre-processing techniques to transform the cardiotocograph waveform data into an optimized format for neural network processing. This includes adaptive baseline calculation algorithms that automatically discount annotated sections when computing baseline values, improving the accuracy and efficiency of pattern detection. The pre-processing component employs technical improvements for handling missing data points and outliers in the raw sensor data streams. Note that this improvement in processing efficiency is beyond merely automating a manual process with a generic computer processer. Rather, as opposed to a generic processor, specific transformation in data formats and discounting of annotated sections provides a technical improvement in processing efficiency of computer algorithms, compared with algorithms not using such features.

The interactive interface provides specific improvements to the computer display of cardiotocograph data by implementing efficient overlay rendering techniques that allow real-time visualization of AI-generated annotations without degrading system performance. The interface uses specialized caching and buffering of waveform data to enable smooth scrolling and zooming of long-duration recordings while maintaining responsiveness. The system implements optimized data structures for storing and accessing the time-series waveform data and corresponding annotations.

The human-in-the-loop architecture is implemented using specialized model update algorithms that can efficiently incorporate user feedback without requiring full model retraining. his includes techniques for weighting and integrating new training examples, selective updating of model parameters, and optimized backpropagation implementations. The system maintains version control of model updates and implements rollback capabilities if performance degrades. In particular, the human-in-the-loop approach allows for a subset of data (e.g., problematic cases, misclassified samples, etc.) to be labeled and incorporated into the model updates. The model is therefore fine-tuned using active learning, reducing the number of required training samples. In contrast, a full retraining approach requires processing all available training data, which can be computationally expensive especially with large datasets. Thus, the human-in-the-loop architecture demands significantly fewer training iterations as it updates the model when necessary rather than entirely, thereby reducing overall CPU/GPU usage, memory allocation, and training time.

The human-in-the-loop approach may use transfer learning or online learning to adjust weights without retraining from scratch. As an example, this approach may fine-tune specific layers in the neural network rather than retraining the entire model by reweighting misclassified/misannotated samples to improve accuracy without modifying well-learned areas. Thus, the human-in-the-loop architecture avoids redundant computations by only refining specific parts of the model, reducing time complexity and computational demands. Further, the human-in-the-loop architecture herein proposed bypasses the need for full hyperparameter tuning, thereby avoiding demanding grid search or Bayesian optimization.

The technical implementation enables real-time processing of streaming cardiotocograph data with latency under 100 ms, allowing for immediate display of AI annotations as new data arrives. This is achieved through optimized neural network architectures, efficient GPU utilization, and specialized data pipeline implementations. The system can process multiple simultaneous data streams from different cardiotocograph devices while maintaining performance.

The disclosure also provides support for a system, comprising: a memory that stores computer executable components, and a processor that executes the computer executable components stored in the memory to: receive manual annotations to a plurality of cardiotocograph datasets via an interactive interface tool, generate, based on the manual annotations to the plurality of cardiotocograph datasets, training data, train a cardiotocograph pattern identification model, using the training data, to identify patterns in cardiotocograph tracings corresponding to defined physiological events associated with hearts of fetuses and uteruses of mothers who correspond to the cardiotocograph tracings during labor, deploy the trained cardiotocograph pattern identification model to identify one or more abnormal patterns in a cardiotocograph waveform sample and generate one or more artificial intelligence (aI)-based annotations for the identified abnormal patterns, and display the one or more aI-based annotations as overlays on the cardiotocograph waveform sample within the interactive interface tool. In a first example of the system, the processor performs further operations comprising: receive one or more user-inputted edits to the one or more AI-based annotations via the interactive interface tool. In a second example of the system, optionally including the first example, the processor performs further operations comprising: update the trained cardiotocograph pattern identification model based on the one or more user-inputted edits. In a third example of the system, optionally including one or both of the first and second examples, the patterns of cardiotocograph data comprise one or more of fetal heart rate (FHR) acceleration, FHR deceleration, a period of FHR variability, and a uterine contraction. In a fourth example of the system, optionally including one or more or each of the first through third examples, the processor receives the manual annotations when the interactive interface tool is in manual annotation mode and wherein the processor displays the one or more AI-based annotations via the interactive interface tool when the interactive interface tool is in display mode. In a fifth example of the system, optionally including one or more or each of the first through fourth examples, a mode of the interactive interface tool is determined according to one or more selector elements, wherein the interactive interface tool is in the manual annotation mode when at least one of the one or more selector elements is toggled on and is in the display mode when the one or more selector elements are toggled off. In a sixth example of the system, optionally including one or more or each of the first through fifth examples, the plurality of cardiotocograph datasets are retrieved from one or more data repositories and are historical. In a seventh example of the system, optionally including one or more or each of the first through sixth examples, the cardiotocograph waveform sample is retrieved from a cardiotocograph device in real-time.

The disclosure also provides support for a method, comprising: obtaining a plurality of cardiotocograph datasets, receiving user-inputted annotations to the plurality of cardiotocograph datasets via an interactive interface tool, wherein the interactive interface tool is configured to display a fetal heart rate (FHR) tracing and a uterine activity (Ua) tracing of a currently displayed waveform sample of the plurality of cardiotocograph datasets in a time-aligned manner, training a cardiotocograph pattern identification model based on the user-inputted annotations, obtaining a first cardiotocograph waveform sample, deploying the trained cardiotocograph pattern identification model to generate one or more model outputs based on the first cardiotocograph waveform sample, wherein the one or more model outputs comprise one or more aI-generated annotations corresponding to sections of the first cardiotocograph waveform sample that correspond to defined waveform patterns, and displaying the first cardiotocograph waveform sample and the corresponding one or more aI-generated annotations within the interactive interface tool. In a first example of the method, the method further comprises: receiving, via the interactive interface tool, one or more user-inputted edits to the one or more AI-generated annotations, wherein the one or more user-inputted edits comprise one or more of changes to the one or more AI-generated annotations, input of new annotations, and deletion of one or more of the one or more AI-generated annotations, and updating the trained cardiotocograph pattern identification model based on the one or more user-inputted edits. In a second example of the method, optionally including the first example, the method further comprises: obtaining a second cardiotocograph waveform sample, deploying the updated cardiotocograph pattern identification model to generate one or more second model outputs, wherein the one or more second model outputs comprise one or more second AI-generated annotations corresponding to sections of the second cardiotocograph waveform sample that correspond to the defined waveform patterns. In a third example of the method, optionally including one or both of the first and second examples, the FHR tracing and the UA tracing are selectable elements, wherein receiving the user-inputted annotations comprises receiving user-inputted selection of sections of the FHR tracing and the UA tracing that correspond to patterns corresponding to physiological events. In a fourth example of the method, optionally including one or more or each of the first through third examples, the method further comprises: generating training data based on the user-inputted annotations, wherein the cardiotocograph pattern identification model is trained to identify the patterns corresponding to physiological events based on the training data. In a fifth example of the method, optionally including one or more or each of the first through fourth examples, the one or more model outputs further comprise one or more baseline values including a baseline FHR value and a baseline uterine tone value. In a sixth example of the method, optionally including one or more or each of the first through fifth examples, the one or more baseline values are determined based on points of the first cardiotocograph waveform sample and the sections of the first cardiotocograph waveform sample that correspond to defined waveform patterns, wherein the one or more baseline values are calculated as an average of the points of the first cardiotocograph waveform sample, discounting the sections of the first cardiotocograph waveform sample that correspond to defined waveform patterns. In a seventh example of the method, optionally including one or more or each of the first through sixth examples, the defined waveform patterns are defined based on the user-inputted annotations and comprise one or more of FHR decelerations, FHR accelerations, periods of FHR variability, and uterine contractions.

The disclosure also provides support for a system, comprising: a processing unit, memory storing instructions executable by the processing unit, an interactive interface tool, and a cardiotocograph pattern identification model, wherein the processing unit is configured to: generate training data based on manual annotations to cardiotocograph data displayed within the interactive interface tool when the interactive interface tool is in manual annotation mode, train the cardiotocograph pattern identification model based on the training data to identify one or more waveform patterns corresponding to defined physiological events, analyze a cardiotocograph waveform sample comprising FHR-Ua tracing data tracked for a defined window of time with the cardiotocograph pattern identification model to identify waveform patterns within the cardiotocograph waveform sample, display the cardiotocograph waveform sample within the interactive interface tool, display the identified waveform patterns as aI-generated annotations overlaid on the displayed cardiotocograph waveform sample within the interactive interface tool, receive user-inputted edits to the aI-generated annotations via the interactive interface tool, and update the cardiotocograph pattern identification model based on the user-inputted edits, wherein the interactive interface tool comprises: an FHR tracing comprising a plot of a corresponding cardiotocograph FHR waveform, a Ua tracing comprising a plot of a corresponding cardiotocograph Ua waveform, a baseline FHR marker element, and a baseline uterine tone marker element. In a first example of the system, the FHR tracing and the UA tracing are selectable elements when the interactive interface tool is in manual annotation mode, wherein selecting a section of a given tracing identifies the section as corresponding to one of the one or more waveform patterns. In a second example of the system, optionally including the first example, the baseline FHR marker element and the baseline uterine tone marker element indicate a baseline value of a corresponding tracing, wherein the baseline value is calculated based on points of the corresponding tracing and sections of the corresponding tracing identified as corresponding to waveform patterns. In a third example of the system, optionally including one or both of the first and second examples, the interactive interface tool is configured to access retrospective cardiotocograph data from one or more data repositories and real-time cardiotocograph data from one or more cardiotocograph devices.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A system, comprising:
a memory that stores computer executable components; and
a processor that executes the computer executable components stored in the memory to:
receive manual annotations to a plurality of cardiotocograph datasets via an interactive interface tool;
generate, based on the manual annotations to the plurality of cardiotocograph datasets, training data;
train a cardiotocograph pattern identification model, using the training data, to identify patterns in cardiotocograph tracings corresponding to defined physiological events associated with hearts of fetuses and uteruses of mothers who correspond to the cardiotocograph tracings during labor;
deploy the trained cardiotocograph pattern identification model to identify one or more abnormal patterns in a cardiotocograph waveform sample and generate one or more artificial intelligence (AI)-based annotations for the identified abnormal patterns; and
display the one or more AI-based annotations as overlays on the cardiotocograph waveform sample within the interactive interface tool.

2. The system of claim 1, wherein the processor performs further operations comprising:
receive one or more user-inputted edits to the one or more AI-based annotations via the interactive interface tool.

3. The system of claim 2, wherein the processor performs further operations comprising:
update the trained cardiotocograph pattern identification model based on the one or more user-inputted edits.

4. The system of claim 1, wherein the patterns of cardiotocograph data comprise one or more of fetal heart rate (FHR) acceleration, FHR deceleration, a period of FHR variability, and a uterine contraction.

5. The system of claim 1, wherein the processor receives the manual annotations when the interactive interface tool is in manual annotation mode and wherein the processor displays the one or more AI-based annotations via the interactive interface tool when the interactive interface tool is in display mode.

6. The system of claim 5, wherein a mode of the interactive interface tool is determined according to one or more selector elements, wherein the interactive interface tool is in the manual annotation mode when at least one of the one or more selector elements is toggled on and is in the display mode when the one or more selector elements are toggled off.

7. The system of claim 1, wherein the plurality of cardiotocograph datasets are retrieved from one or more data repositories and are historical.

8. The system of claim 1, wherein the cardiotocograph waveform sample is retrieved from a cardiotocograph device in real-time.

9. A method, comprising:
obtaining a plurality of cardiotocograph datasets;
receiving user-inputted annotations to the plurality of cardiotocograph datasets via an interactive interface tool, wherein the interactive interface tool is configured to display a fetal heart rate (FHR) tracing and a uterine activity (UA) tracing of a currently displayed waveform sample of the plurality of cardiotocograph datasets in a time-aligned manner;
training a cardiotocograph pattern identification model based on the user-inputted annotations;
obtaining a first cardiotocograph waveform sample;
deploying the trained cardiotocograph pattern identification model to generate one or more model outputs based on the first cardiotocograph waveform sample, wherein the one or more model outputs comprise one or more AI-generated annotations corresponding to sections of the first cardiotocograph waveform sample that correspond to defined waveform patterns; and
displaying the first cardiotocograph waveform sample and the corresponding one or more AI-generated annotations within the interactive interface tool.

10. The method of claim 9, further comprising:
receiving, via the interactive interface tool, one or more user-inputted edits to the one or more AI-generated annotations, wherein the one or more user-inputted edits comprise one or more of changes to the one or more AI-generated annotations, input of new annotations, and deletion of one or more of the one or more AI-generated annotations; and
updating the trained cardiotocograph pattern identification model based on the one or more user-inputted edits.

11. The method of claim 10, further comprising:
obtaining a second cardiotocograph waveform sample;
deploying the updated cardiotocograph pattern identification model to generate one or more second model outputs, wherein the one or more second model outputs comprise one or more second AI-generated annotations corresponding to sections of the second cardiotocograph waveform sample that correspond to the defined waveform patterns.

12. The method of claim 9, wherein the FHR tracing and the UA tracing are selectable elements, wherein receiving the user-inputted annotations comprises receiving user-inputted selection of sections of the FHR tracing and the UA tracing that correspond to patterns corresponding to physiological events.

13. The method of claim 12, further comprising generating training data based on the user-inputted annotations, wherein the cardiotocograph pattern identification model is trained to identify the patterns corresponding to physiological events based on the training data.

14. The method of claim 9, wherein the one or more model outputs further comprise one or more baseline values including a baseline FHR value and a baseline uterine tone value.

15. The method of claim 14, wherein the one or more baseline values are determined based on points of the first cardiotocograph waveform sample and the sections of the first cardiotocograph waveform sample that correspond to defined waveform patterns, wherein the one or more baseline values are calculated as an average of the points of the first cardiotocograph waveform sample, discounting the sections of the first cardiotocograph waveform sample that correspond to defined waveform patterns.

16. The method of claim 9, wherein the defined waveform patterns are defined based on the user-inputted annotations and comprise one or more of FHR decelerations, FHR accelerations, periods of FHR variability, and uterine contractions.

17. A system, comprising:
a processing unit;
memory storing instructions executable by the processing unit;
an interactive interface tool; and
a cardiotocograph pattern identification model, wherein the processing unit is configured to:
generate training data based on manual annotations to cardiotocograph data displayed within the interactive interface tool when the interactive interface tool is in manual annotation mode;
train the cardiotocograph pattern identification model based on the training data to identify one or more waveform patterns corresponding to defined physiological events;
analyze a cardiotocograph waveform sample comprising FHR-UA tracing data tracked for a defined window of time with the cardiotocograph pattern identification model to identify waveform patterns within the cardiotocograph waveform sample;
display the cardiotocograph waveform sample within the interactive interface tool;
display the identified waveform patterns as AI-generated annotations overlaid on the displayed cardiotocograph waveform sample within the interactive interface tool;
receive user-inputted edits to the AI-generated annotations via the interactive interface tool; and
update the cardiotocograph pattern identification model based on the user-inputted edits, wherein the interactive interface tool comprises:
an FHR tracing comprising a plot of a corresponding cardiotocograph FHR waveform;
a UA tracing comprising a plot of a corresponding cardiotocograph UA waveform;
a baseline FHR marker element; and
a baseline uterine tone marker element.

18. The system of claim 17, wherein the FHR tracing and the UA tracing are selectable elements when the interactive interface tool is in manual annotation mode, wherein selecting a section of a given tracing identifies the section as corresponding to one of the one or more waveform patterns.

19. The system of claim 17, wherein the baseline FHR marker element and the baseline uterine tone marker element indicate a baseline value of a corresponding tracing, wherein the baseline value is calculated based on points of the corresponding tracing and sections of the corresponding tracing identified as corresponding to waveform patterns.

20. The system of claim 18, wherein the interactive interface tool is configured to access retrospective cardiotocograph data from one or more data repositories and real-time cardiotocograph data from one or more cardiotocograph devices.
